(19) United States
(12) Patent Application Publication
(10) Pub. No.: US 2009/0148011 A1
(43) Pub. Date: Jun. 11, 2009

(54) IN-SITU DATA COLLECTION ARCHITECTURE FOR COMPUTER-AIDED DIAGNOSIS

(75) Inventor: Luyin Zhao, White Plains, NY (US)

Correspondence Address: PHILIPS INTELLECTUAL PROPERTY & STANDARDS, P.O. BOX 3001, BRIARCLIFF MANOR, NY, 10510, US

(73) Assignee: Konnklike Philips Electronics, N.V., Eindhoven (NL)

(21) Appl. No.: 11/719,793

(22) PCT Filed: Nov. 16, 2005
(86) PCT No.: PCT/IB05/53779

§ 371 Date: May 21, 2007

Related U.S. Application Data
(60) Provisional application No. 60/629,753, filed on Nov. 19, 2004.
(60) Provisional application No. 60/659,363, filed on Mar. 7, 2005.

(51) Int. Cl. G06K 9/00; A61B 5/05

(52) U.S. Cl. 382/128; 600/407

(57) ABSTRACT

Automated diagnostic decision support (104) in the imaging of potentially malignant lesions is distributed and streamlined to protect patient confidentiality and to lower bandwidth and transaction costs. At a client hospital site (108a, 108b), a software agent (132) monitors a database and responsively accesses an image of a lesion and ground truth that the lesion is malignant/benign (S310-S330). After computing at least one feature of the lesion based on the image (S340, S350), the software agent transmits the feature(s) and ground truth externally from the hospital, to a central diagnostic decision support server (S360, S370). When a client hospital site needs automatic diagnostic support, the lesion feature(s) of the new patient are likewise extracted and transmitted to the external server in a query message (S440). The classifier located on the server will return a diagnosis (benign/malignant) and a confidence level (S450, S460).

The present invention relates to automated diagnosis support and, more particularly, to focused, efficient data-collection for automated diagnosis support.

Healthcare diagnosis decision support systems or computer-aided diagnosis (CAD) systems are used to classify unknown lesions or tumors detected in digital images into different categories, e.g., malignant or benign. Usually, machine-learning technologies, such as a decision tree and neural network, are utilized to build classifiers based on a large number of known cases with ground truth, i.e., cases for which the diagnosis has been confirmed by pathology. The classifier bases its diagnosis on a computational structure built from known cases and inputted features for the unknown tumor case. The classifier output indicates the estimated nature (e.g., malignant/benign) of the unknown tumor and optionally a confidence value. As the precision of medical imaging facilities improves to detect very small tumors, and as the number of digital images to be processed increases this type of CAD becomes increasingly important as a tool to assist physicians. The computer-produced classification is considered a second opinion to a physician in order to raise the accuracy and confidence associated with diagnosis.

One of the major problems in CAD is the difficulty in obtaining enough data or known cases to train the computer. Aside from technical difficulties, there are many reasons, such as unwillingness by hospitals to disclose patient images, high cost to access of such data, or other social/political reasons. The largest data set used by past research projects contains merely a few hundred cases.

This problem becomes critical because the reliability, trustworthiness and future Federal Drug Administration (FDA) approval criteria for CAD solutions are largely dependent upon the number of training cases used to build CAD software and on the degree to which such cases are representative.

Therefore, it is proposed herein to distribute data acquisition in an architecture that affords continuous and incremental training for CAD solutions. Only the data necessary is acquired from the hospital, rather than the whole digital images.

The present inventor has realized that building a reliable CAD solution only needs more image features (e.g., measures of circularity, mean gray value, angularity, margin, shape, density, spiculation, etc.) and ground truth associated with the lesion. Other patient-sensitive data, such as patient name, date of birth, and even the whole digital image, that are conventionally considered prerequisites for CAD and are difficult to obtain from clinical sites, are not actually necessary.

Using distributed computing technologies, lesion features and ground truth are derived within the boundaries of the clinical site, and this information, in and of itself, may be disclosed to a central CAD server without the need for any further disclosure. This differs from the traditional paradigm of acquiring images from clinical sites and then doing feature extraction. The change from post-processing to pre-processing makes it easier to obtain useful information for building CAD solutions, while minimizing the risk and difficulty of working on real patient images.

In one aspect, a method for collecting medical data involves capturing, at a client site, an image of a lesion of a medical subject at the client site. From the captured image, at least one feature of the lesion is derived. The at least one feature and ground truth that the lesion is either malignant or benign is transmitted by the client site to a server disposed externally to the client site.

In another aspect, a data-collecting device located at a client site receives ground truth that a lesion of a medical subject is either malignant or benign. The device pairs the received ground truth with at least one feature characteristic of the lesion computed from an image of the lesion. The pair is transmitted to a server disposed externally to the client site.

In yet another aspect, a server has a receiver for receiving, from any of plural client sites, a respective pair comprising (a) ground truth that a lesion is either malignant or benign; and (b) at least one feature of a lesion derived from an image of the lesion. The server also includes a diagnostic support processor for incremental training based on the received pair. The sites are located externally from each other and from the server.

As a further aspect, a computer software product for collecting medical data and located at a client site is embedded within a medium readable by a processor. The product contains instructions executable to monitor a database at the client site. Further instructions obtain, from the database responsive to the monitoring, an image of a lesion of a medical subject and ground truth that the lesion is either malignant or benign. The product also includes instructions for outputting, for transmission to a server disposed externally to the client site, the accessed ground truth and at least one feature of the lesion derived from the accessed image.

Details of the invention disclosed herein shall be described with the aid of the figures listed below, wherein:

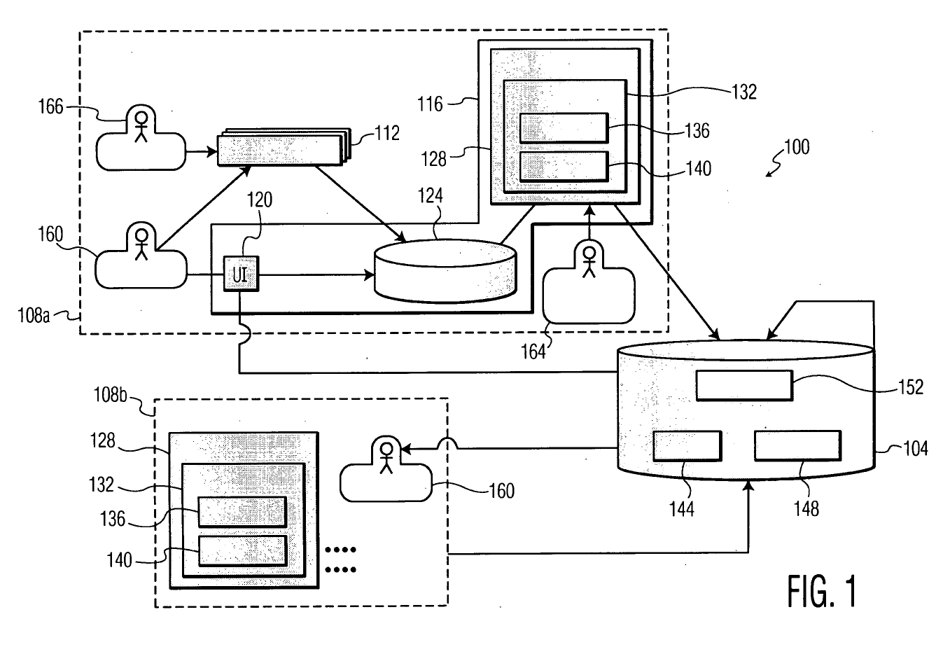

FIG. 1 depicts a CAD input-information collection system according to the present invention;

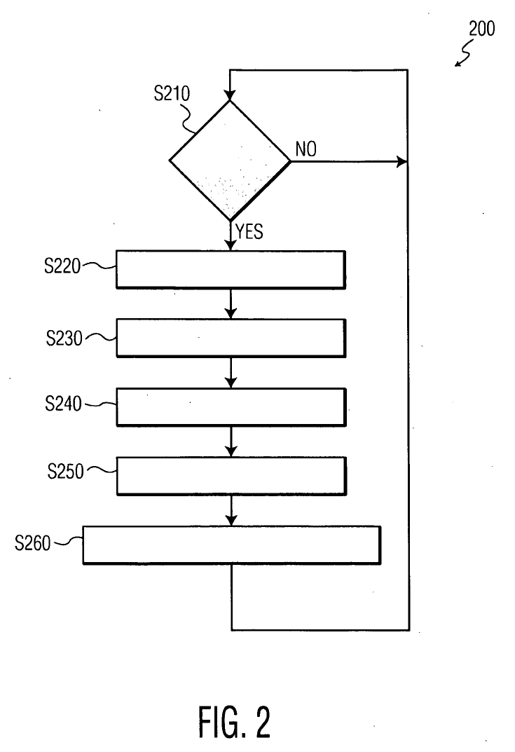

FIG. 2 is a flowchart of a client-database building sub-process according to the present invention;

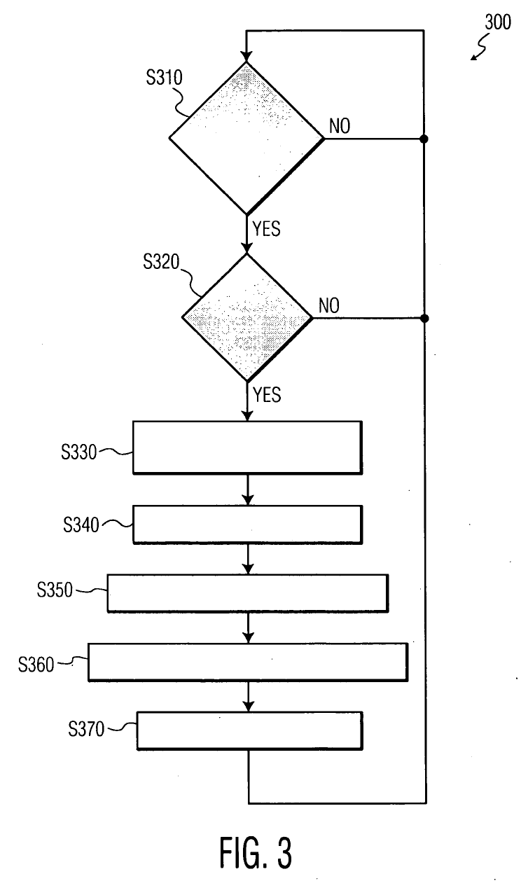

FIG. 3 is a flowchart of software-agent processing according to the present invention; and

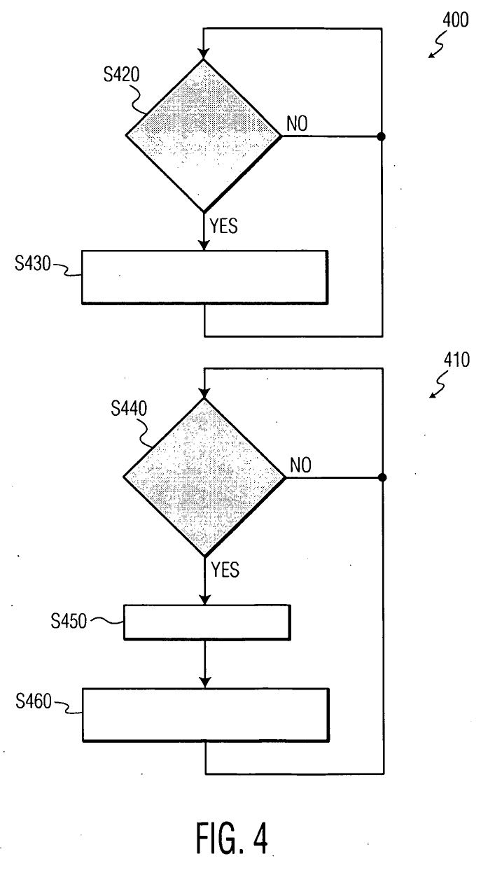

FIG. 4 is a pair of flowcharts of server processing according to the present invention.

FIG. 1 depicts, by way of illustrative and non-limitative example, a CAD input-information collection system 100 according to the present invention. The system 100 includes a diagnostic decision support server 104 and client hospitals (or “client sites”) 108a, 108b. Only one client hospital may be included or more than two client hospitals (not shown), and preferably many more than two client hospitals.

Within the client hospital 108a are an imaging device 112 and a data collecting device 116, these devices being connected. The imaging by the imaging device 112 may be of any type, e.g., ultrasound, computed tomography (CT), magnetic resonance imaging (MRI).

The data collecting device 116 includes a user interface (UI) 120, a patient database 124, and a memory 128 that contains a software agent 132. The memory 128 preferably includes random access memory (RAM) and read-only memory (ROM) in any of their various forms.

The software agent 132 has a segmentation algorithm 136 and a feature extraction algorithm 140.

For receiving transmissions from the client hospitals 108a, 108b, the server 104 has a receiver 144. Results of processing by the processor 148 are sent to respective clients 108a, 108b by the transmitter 152.

A radiologist or other medical professional 160 operates the data-collecting device 116, and approval by a hospital authority or administrator 164 may be needed to authorize the movement of information from the hospital 108a, 108b to the external server 104.

FIG. 2 shows an example of a client-database building sub-process 200 according to the present invention. When a lesion or tumor of a new patient 166 is imaged on the imaging device 112 (steps S210, S220), the radiologist reviewing the output makes a diagnosis on whether the lesion is malignant or benign. The diagnosis can be made by expert judgment, i.e., benign lung nodules do not grow in a two-year period, or based on biopsy or surgery. The radiologist 160 may also draw upon CAD support from the server 104 in arriving at a diagnosis, as it will be discussed in more detail further below. Any of these techniques can be used alone or in combination. The acquired or captured image of the lesion is stored in the patient database 124. This may occur before or after the diagnosis (steps S230, S240). It is assumed herein that information of the new patient 166 is ultimately transmitted to the server 104 only once.

To add the new patient 166 as a case that is suitable for use in building the automated diagnostic decision support system, ground truth about the lesion is preferably acquired first. Ground truth typically entails information acquired independently of the imaging to confirm or disconfirm the diagnosis by pathology. Thus, for example, surgery or biopsy may bring a quick resolution. The non-development of the tumor over time (e.g., two years) may also yield ground truth of benignity.

When ground truth is obtained (step S250), the radiologist or other medical practitioner 160 may operate the data collecting device 116, via the user interface, to store the ground truth in the patient database 124. The ground truth is preferably stored together with a location in the image of the lesion (step S260). The image itself typically would have already been stored previously.

FIG. 3 demonstrates one example of software-agent processing 300 according to the present invention. The software agent 132 may function autonomously to selectively extract information from the database 124 for transmission to the server 104, albeit optionally subject to authorization from the hospital administrator 164. A charging or billing application may be launched at this point if provision of the input data for the server 104 is not free.

In one embodiment, the software agent 132 continuously monitors the database 124 to detect whenever ground truth is added (step S310). Alternatively, monitoring is such that the software agent 132 is notified when ground truth is added. The notification may be performed periodically or after a predetermined number of ground truth additions, or according to any other criteria such as tightness of storage in the database.

When the software agent 132 is ready to process information from the database 124, the data-collecting device 116 may contact the hospital authority 164, as by a user interface (not shown). If authorization is given (step S320), the device 116 or the hospital authority 164 may launch a billing application. In any event, the device 116 gains access to the ground truth and the image of the lesion (step S330). Alternatively, the device 116 may access this information for any number of lesions of respective patients. However, regardless of the protocol, normally a single ground truth is accessed for a given lesion of a given patient. In the rare event of the ground truth changing over time due to changing pathology, the software agent 132 may augment the pair to be transmitted to the server 104 with an indication that this pair updates a previous pair.

As a general measure to preserve the integrity of the system 100, the software agent 132 may flag the database entry being accessed. Thus, if the patient 166 leaves the hospital 108a, 108b for another hospital, the transferred patient records will indicate that the patient's information has already been inputted to build diagnostic decision support in the server 104, thereby preventing a double input for the same lesion.

The agent 132 first uses the segmentation algorithm 140 to segment the lesion in the image (step S340), thereby isolating it from its background and/or other structures in the image. Methods of regularizing an image or otherwise segmenting objects within an image are well-known in the medical imaging field.

Next, the extraction algorithm 136 computes one or more features to thereby extract them from the image of the lesion (step S350). One such feature might be, for example, a measure of angularity. The extracted features may belong to a particular set of kinds or categories of features, which may or may not vary with each processed lesion. Automated feature extraction may be effected by techniques that are, likewise, well-known in the medical imaging field.

At least one, and preferably all, of the features computed for the lesion are paired with the ground truth for transmission to the server 104 (step S360). Any information from the database 124, or from any other source in the hospital 108a, 108b, that might serve to identify the new patient 166, is excluded from the transmission. This safeguards patient confidentiality. Bandwidth is conserved by limiting the transmission to such a pair, or pairs, thereby reducing processing cost. In addition, the continuous and automatic nature of the processing reduces the transaction burden, thus further reducing cost.

The software agent 132 outputs the pair(s) for transmission or more actively participates in the transmitting (step S370). The pair, or preferably pairs, forms the payload of the message or packet being transmitted from the hospital 108a, 108b to the server 104.

Generally, no other patient information is needed at the server. An exception for which additional information might be desirable is in the case where the new patient 166 has more than one lesion to be investigated. The software agent 132 will handle the two or more lesions separately but may indicate that the pairs being transmitted to the server 104 pertain to the same patient. This indication may come, for example, from the arrangement of the data in the message payload. For example, if multiple pairs are typically sent in the same transmission in the order of ground truth, feature(s), ground truth, feature(s), . . . , two tumors of the same patient may be represented in the order of ground truth, ground truth, feature(s), feature(s). Alternatively, the multiple pairs of the same patient may be otherwise linked without changing the order of fields in the payload. Other information may also be added to the message, in this case of multiple tumors of the same patient, or in the case of a single tumor, although any information that would identify a patient is not needed.

FIG. 4 presents flowcharts exemplary of a training sub-process 400 and of a query sub-process 410. When the server 104 receives a transmitted message (step S420), the server adds the ground truth, feature(s) pair, or each one, as a new case. The server 104 incrementally trains using the new case(s) (step S430). For example, the server 104 trains using a first new case, (i.e.,)? and again trains using a second new case, etc. Alternatively, the server 104 may train using all new cases received in the transmission from the hospital 108a, 108b, and then train again based on any subsequently received transmission. If multiple pairs are in the message payload, the server 104 preferably also notes any indication, as by the ordering of the fields, that a plurality of cases pertain to the same patient.

Upon receiving a request for automated diagnostic support (step S440), from the hospital 108a, 108b, a classifier (not shown) in the processor 148 prepares a response (S450). The request may be accompanied by the image of the tumor, and any other pertinent information not identifying the patient. For example, the request may contain features of the lesion, extracted in the manner described above or in any other known and suitable manner. These features may be included instead of, or in addition to, in the image of the tumor. The response would normally include a diagnosis, and perhaps an associated confidence level associated with the diagnosis. The response might also include what the classifier determines to be images of similar cases and their respective ground truths. In one embodiment, these images of similar cases may have accompanied incoming ground truth/feature(s) pairs. The response is sent back to the requesting client site 108a, 108b (step S460) and is presented over UI 120 to the radiologist 160. The UI 120 handling the request and response may be the same user interface or a user interface different from that used by the radiologist 160 in entering ground truth information.

While there have been shown and described and noted fundamentally novel features of the invention as applied to preferred embodiments thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps that perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

1. A method for collecting medical data, comprising the following acts:
(a) capturing, at a client site, an image of a lesion of a medical subject at the client site (S220);
(b) deriving, from the captured image, at least one feature of the lesion (S350); and
(c) transmitting, by the client site to a server disposed externally to the client site, one or more features derived in act (b) and ground truth that the lesion is either malignant or benign (S360, S370).

2. The method of claim 1, further comprising pairing the ground truth to said one or more features to form, optionally in combination with any other pairing of ground truth to one or more associated features according to the method of claim 1, the payload of a message to be transmitted in the transmitting (S360, S370).

3. The method of claim 2, wherein said server is common to a plurality of client sites each respectively performing the capturing and transmitting of claim 1 (108a, 108b).

4. The method of claim 3, further comprising:
receiving, by the server from a client site of the plural client sites, the transmitted payload (S420);
training, on the server, based on the received payload (S430);
sending, by the server to a destination client site of the plural client sites, diagnostic decision support information (S460);
receiving, by the destination client site, the sent diagnostic decision support information (120); and
presenting, at said destination client site, the received diagnostic decision support information (120).

5. The method of claim 1, wherein said deriving is performed at the client site (132), said method further comprising deriving, at the client site, said ground truth (S260).

6. The method of claim 1, comprising:
making a diagnosis that the lesion is either malignant or benign (S240); and
confirming, by pathology, the diagnosis as either valid or invalid, thereby creating said ground truth (S250).

7. The method of claim 1, further including the act of excluding, from the transmitting, any information identifying said medical subject (S330).

8. A data collecting device (116) located at a client site and configured for:
receiving ground truth that a lesion of a medical subject is either malignant or benign (S260);
pairing the received ground truth with at least one feature characteristic of the lesion computed from an image of the lesion (S350, S360); and
transmitting the pair to a server disposed externally to the client site (S370).

9. The device of claim 8, further comprising:
a user interface by which to input the ground truth (120); and
the same or different user interface (120), said device being configured for receiving, from said server, over said same or different user interface diagnostic decision support information (S460).

10. The device of claim 8, wherein said pairing forms, optionally in combination with any other pairing of ground truth to one or more associated features according to the method of claim 8, the payload of a message sent in said transmitting (S450, S460).

11. The device of claim 8, further comprising a database for saving a location of said lesion in the image and the respective ground truth for the lesion (124).

12. The device of claim 11, further comprising:
a memory (128); and
in the memory, a software agent configured for, optionally subject to authorization (164), accessing the database to compute said at least one feature and to retrieve said respective ground truth (132).

13. The device of claim 12, wherein said agent includes:
a segmentation algorithm for segmenting the lesion in the image (140); and
a feature extraction algorithm for computing said at least one feature by extracting, from the segmented lesion in said image, said at least one feature (136).

14. The device of claim 8, configured for excluding, from the transmitting, any information identifying said medical subject (S330).

15. The device of claim 8, further configured for computing said at least one feature, said at least one feature comprising a measure of at least one of: circularity, mean gray value, angularity, margin, shape, density and speculation (S350).

16. An apparatus located at the client site and comprising:
the device of claim 8 (116); and
an imaging device configured for capturing said image from the medical subject at the client site (112).

17. A system for collecting medical data, comprising:
said server of claim 8 (104); and
a plurality of the devices of claim 8 located at respective client sites that are each clients of said server (108a, 108b).

18. The system of claim 17, wherein at least one of the plural devices is further configured for:
in performing the pairing, forming, into a message payload, the ground truth to be transmitted and the at least one feature to be transmitted; and
in performing said transmitting, sending the message payload (S360, S370).

19. A server (104) comprising:
a receiver for receiving, from any of the plural client sites, a respective pair comprising (a) ground truth that a lesion is either malignant or benign; and (b) at least one feature of a lesion derived from an image of the lesion (144); and
a diagnostic support processor for incrementally training (S430), based on the received pair, said sites being located externally from each other and from the server (148).

20. The server of claim 19, further comprising a transmitter for sending, to a destination client site of the plural client sites, diagnostic decision support information (152).

21. The server of claim 19, wherein said respective pair forms, optionally in combination with any other pairing of ground truth to one or more associated features according to claim 19, the payload of a message transmitted from one of the plural client sites, such that the receiving of the respective pair or pairs receives the message payload (S360, S370).

22. A computer software product for collecting medical data (132), said product being located at a client site and embedded within a medium (128) readable by a processor, said product comprising instructions executable to perform acts comprising:
monitoring a database at the client site (S310);
responsive to said monitoring, accessing, from the database, an image of a lesion of a medical subject and ground truth that the lesion is either malignant or benign (S320, S330); and
outputting, for transmission to a server disposed externally to the client site, the accessed ground truth and at least one feature of the lesion derived from the accessed image (S360, S370).

23. The product of claim 22, comprising instructions executable to perform the act of pairing: (a) the ground truth to be transmitted; and (b) said at least one feature to be transmitted, the pair forming, optionally in combination with any other pairing of ground truth to one or more associated features according to claim 22, the payload of a message to be transmitted in said transmission (S360, S370).
